(19) United States
(12) Patent Application Publication
(10) Pub. No.: US 2004/0053391 A1
(43) Pub. Date: Mar. 18, 2004

(54) Renal cell carcinoma-antigen G250-derived peptides that elicit both CD4+ and CD8+ T-cell responses

(75) Inventors: Joost Lambert Max Vissers, Nijmegen (NL); Ingrid Jolanda Monique De Vries, Nijmegen (NL); Egbert Oosterwijk, Nijmegen (NL); Carl Gustav Figdor, Nijmegen (NL); Gosse Jan Adema, Nijmegen (NL)

Correspondence Address: William H. Logsdon, WEBB ZIESENHEIM LOGSDON ORKIN & HANSON, P.C., 700 Koppers Building, 436 Seventh Avenue, Pittsburgh, PA, 15219-1818, US

(73) Assignee: Katholieke Universiteit Nijmegen, Nijmegen (NL)

(21) Appl. No.: 10/241,814

(22) Filed: Sep. 11, 2002

(51) Int. Cl. C12N 9/64

(52) U.S. Cl. 435/226

(57) ABSTRACT

The present invention relates to immunogenic peptides that can be used to elicit an immune response in a human or animal against a tumor, in particular against an immunogenic tumor. The peptides are derived from the G250 tumor antigen that is frequently expressed on immunogenic tumors, such as renal cell carcinoma. The particular peptides are selected for their capability to elicit both CD4⁺ and CD8⁺ T-cell responses against cells expressing the G250 antigen.

### FIELD OF THE INVENTION

The present invention relates to peptides that can be used to elicit an immune response in a human or animal. In particular, the invention relates to immunogenic peptides that can be used to elicit an immune response in a human or animal against a tumor, in particular against an immunogenic tumor. More in particular, the invention relates to peptides derived from a tumor antigen that can be used in the immunotherapy of renal cell carcinoma

### BACKGROUND OF THE INVENTION

According to one way of classifying tumors, a distinction is made between immunogenic and non-immunogenic tumors. Immunogenic tumors can be described as tumors that express certain antigens on their surface. Examples thereof include melanoma, renal cell carcinoma or other tumors of the kidneys, as well as tumors of the prostate, head and/or neck, colon, stomach, and bladder. The finding that immunogenic tumors express specific antigens on their surface has opened up the possibility of treating such tumors using immunological methods.

Renal cell carcinoma (RCC) is also a relatively immunogenic tumor. In a fair number of RCC patients “spontaneous” partial or complete remissions have been observed and some forms of immunotherapy have been shown to increase the reactivity of the immune system against RCC (Freed et al., 1977, J. Urol. 118: 538-542; Marcus et al., 1993, J. Urol, 150: 463-466; Gleave et al., 1998, N. Engl. J. Med. 338: 1265-1271). However, only a few specific cytotoxic T cell (CTL) lines for autologous RCC have been identified so far (Koo et al., 1991, J. Immunother. 10: 347-354; Finke et al., 1992, J. Immunother. 11: 1-11; Schendel et al., 1993, J. Immunol. 151: 4209-4220; Gaugler et al., 1996, Immunogenetics 44: 323-330; Brandle et al., 1996, J. Exp. Med. 183: 2501-2508). One RCC-specific antigen that was defined by such CTL's is RAGE-1, which is expressed in only 2% of primary RCCs and is silent in normal tissue, except retina (Gaugler et al., supra). A second CTL-defined RCC antigen appeared to be a mutated HLA-A2 protein (Brandle et al., supra).

For effective treatment of RCC patients with immunotherapy, high antigen expression in all of the RCCs is a first prerequisite. We have previously demonstrated that monoclonal (mAb) G250 recognizes an RCC-associated antigen expressed on the surface of 85% of RCCs but not on normal tissue (Oosterwijk et al., 1986, Int. J. Cancer 38: 489-494). In addition, G250 expression can be detected on the cell surface of colon, ovarian and cervical carcinomas. Analysis of normal tissues indicated that the reactivity of the G250 mAb is limited to some gastric mucosal cells and to cells of the larger bile duct. The staining observed in these normal tissues is relatively weak and cytoplasmic in nature (Oosterwijk et al., supra; Pastorek et al., 1994, Oncogene 9: 2877-2888; Saarnio et al., 1998, Am, J. Pathol. 153: 279-285). Clinical studies in RCC patients demonstrated exclusive targeting of radiolabeled mAB G250 to RCC (Oosterwijk et al., 1995, Semin. Oncol. 22: 34-41).

Isolation of the cDNA encoding the RCC-associated antigen recognized by the G250 mAb and analysis of the deduced amino acid sequence showed that the G250 protein is a transmembrane protein identical to the previously described tumor-associated antigen MN/CA IX that was identified in cervical carcinoma (Grabmaier et al., 2000, Int. J. Cancer 85: 865-870; Pastorek et al., supra; WO 93/18152; EP-A 1123 387). Sequence comparisons of the G250/MN/CA IX gene with the RCC-derived cDNA of G250 demonstrated that the G250/MN/CA IX protein in RCC is non-mutated (Opavsky et al., 1996, Genomics 33: 480-487; Grabmaier et al., supra). The G250 antigen is therefore a widely expressed RCC-associated antigen and as such constitutes a promising target for specific immunotherapy in RCC patients.

Previously, we demonstrated that the RCC-associated antigen G250 comprises within its sequence an HLA-A2.1-restricted epitope from amino acids 254 through 262, which can be recognized by CD8+CTL's (Vissers et al., 1999, Cancer Res. 59: 5554-5559; WO 01/98363). Next to CTL's, the importance of T-helper cells in antitumor immunity has been clearly demonstrated in several murine tumor models (Schild et al., 1987, Eur. Immunol. 17: 1863-1866; Romerdahl et al., 1988, Cancer Res. 48: 2325-2328; Hung et al., 1998, J. Exp. Med. 188: 2357-2358). Ossendorp et al. (1998, J. Exp. Med, 187: 693-702) demonstrated that CD4⁺ T-helper cells are needed for optimal induction of antitumor-specific CTL's, most likely by activating professional APC's. In addition, T-helper cells participate in the effector phase of the immune response by recruiting and activating macrophages and eosinophils (Hung et al., supra; Greenberg, 1991, Adv. Immunol. 49: 281-355). Therefore, vaccines designed to treat cancer preferably should elicit both CD4⁺ and CD8⁺ T-cell responses to epitopes derived from tumor-associated antigens.

Thus, it is an object of the invention to provide for G250-derived peptides containing epitopes that elicit both CD4⁺ and CD8⁺ T-cell responses and that may effectively be used in (vaccines for) immunotherapy of RCC and other tumors that express the G250 antigen.

### DESCRIPTION OF THE INVENTION

In the research leading up to the present invention, the immunogenicity of the RCC-associated antigen G250 was investigated using the reversed immunology approach. By this approach, a number of possible peptides based upon the amino acid sequence of the G250 protein were developed. In particular, it was found that of these, the G250 amino acid sequence from 254 to 262 is an HLA-A2.1-restricted CTL epitope that is both naturally processed and immunogenic, and thus can be used in the immune therapy of cancers, in particular of cancers expressing the G250 protein. In further research we investigated whether the G250 antigen, besides a CTL response, also can mount a T-helper response. Using computer-aided prediction programs and DC's loaded with synthetic G250-derived peptides, we induced HLA-DR-restricted T-helper cells against the G250-derived peptide from amino acids 249-268 that also recognize naturally processed G250 protein Surprisingly, the previously identified CTL epitope in the G250 amino acid sequence from 254 to 262 is fully comprised within the T-helper cell epitope in the G250 amino acid sequence from 249 to 268. The latter may thus be used to derive peptides that induce both CTL and T-helper responses for use in the immune therapy of cancers expressing the G250 protein.

Peptides of the Invention

In a first aspect, the invention relates to a peptide comprising the amino acid sequence of SEQ ID NO. 15 or an amino acid sequence with at most 1, 2, 3 or 4 amino acid replacements with respect to the amino acid sequence of SEQ ID NO. 15. In an alternative embodiment, the peptide consists of the amino acid sequence of SEQ ID NO. 15 or an amino acid sequence with at most 1, 2, 3 or 4 amino acid replacements with respect to the amino acid sequence of SEQ ID NO. 15.

Preferably, the peptide of the invention comprises the amino acid sequence of SEQ ID NO. 15 and 1 to 5 additional amino acids from SEQ ID NO. 12 or an amino acid sequence with at most 1, 2, 3, 4, or 5 amino acid replacements with respect to the amino acid sequence of SEQ ID NO. 15 and the 1 to 5 additional amino acids from SEQ ID NO. 12. Alternatively, the peptide of the invention consists of the amino acid sequence of SEQ ID NO. 15 and 1 to 5 additional amino acids from SEQ ID NO. 12 or an amino acid sequence with at most 1, 2, 3, 4, or 5 amino acid replacements with respect to the amino acid sequence of SEQ ID NO. 15 and the 1 to 5 additional amino acids from SEQ ID NO. 12.

In a preferred peptide of the invention, the amino acid replacements are conservative replacements as defined herein below. Particularly preferred amino acid replacements in the amino acid sequence of SEQ ID NO. 12 are L, P, A, F, W or M in position 4, M in position 7 and/or L, P, A, F, W or M in position 14 The peptides of the invention preferably is a peptide other than a human G250 protein having the amino acid sequence of SEQ ID NO. 16. Similarly, the peptides of the invention preferably do not include any of the (G250/MN/CA IX) peptides or fragments thereof disclosed as such or as encoded by nucleotide sequences disclosed in any of Grabmaier et al., supra; Pastorek et al., supra; WO 93/18152; EP-A 1 123 387; Opavsky et al., supra; Vissers et al., supra; and WO 01/98363).

As used herein, the term “peptide” is understood to include both oligopeptides as well as polypeptides, which are also referred to as proteins. The peptides of the invention contain an epitope that specifically recognized by both MHC class I and II molecules. The peptides of the invention are thus capable of bind the groove or cleft of an MHC class II molecule. The peptides of the invention will therefore typically comprise at least about 9, 10, 11, 12, 15, or 18 residues. In certain embodiments the peptides will not exceed about 150, 100 or 50 residues and typically will not exceed about 20 residues. In other embodiments the peptides of the invention may be (much) larger polypeptides or protein comprising the MHC class I and II epitopes, e.g. as part of a fusion protein. Thus, a wide range of peptide sizes may be used in the present invention.

Particularly when the peptides of the invention are relatively short, the peptides can be readily synthesized using known methods. For example, the peptides can be synthesized by the well-known Merrifield solid-phase synthesis method in which amino acids are sequentially added to a growing chain. See Merrifield (1963), J. Am. Chem. Soc. 85.2149-2156; and Atherton et al., “Solid Phase Peptide Synthesis,” IRL Press, London, (1989). Automatic peptide synthesizers are commercially available from numerous suppliers, such as Applied Biosystems, Foster City, Calif. Additional synthetic approaches for preparing the peptides of the invention are described in the Examples herein.

Alternatively, the peptides of the invention may be larger polypeptides or proteins comprising the epitopes of the invention Such larger polypeptides are preferably prepared using well-known recombinant techniques in which a nucleotide sequence encoding the polypeptide of interest is expressed in cultured cells such as described in Ausubel et al., Current Protocols in Molecular Biology, Greene Publishing and Wiley-Interscience, New York (1987) and in Sambrook and Russell (2001) “Molecular CloningL A Laboratory Manual” (3^{rd} edition), Cold Spring Harbor Laboratory, Cold Spring Harbor Laboratory Press, New York, both of which are incorporated herein by reference in their entirety.

Typically, nucleic acids encoding the desired polypeptides are used in expression vectors. The phrase “expression vector” generally refers to nucleotide sequences that are capable of affecting expression of a gene in hosts compatible with such sequences. These expression vectors typically include at least suitable promoter sequences and optionally, transcription termination signals. Additional factors necessary or helpful in effecting expression may also be used as described herein. DNA encoding the polypeptides of the present invention will typically be incorporated into DNA constructs capable of introduction into and expression in an in vitro cell culture. Specifically, DNA constructs will be suitable for replication in a prokaryotic host, such as bacteria, e.g., E. coli, or maybe introduced into a cultured mammalian, plant, insect, yeast, fungi or other eukaryotic cell lines.

DNA constructs prepared for introduction into a particular host will typically include a replication system recognized by the host, the intended DNA segment encoding the desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic and eukaryotic promoters well known in the art. See, e.g., Sambrook and Russell (2001, supra). The transcriptional regulatory sequences will typically include a heterologous enhancer or promoter that is recognized by the host. The selection of an appropriate promoter will depend upon the host, but promoters such as the trp, lac and phase promoters, tRNA promoters and glycolytic enzyme promoters are known and available. See, e.g., Sambrook and Russell (2001, supra).

Conveniently available expression vectors which include the replication system and transcriptional and translational regulatory sequences together with the insertion site for the polypeptide-encoding segment may be employed. Examples of workable combinations of cell lines and expression vectors are described in Sambrook and Russell (2001, supra). For example, suitable expression vectors may be expressed in, e.g., insect cells, e.g., Sf9 cells, mammalian cells, e.g., CHO cells and bacterial cells, e.g., E. coli.

It will be understood that the peptides of the invention may be modified to provide a variety of desired attributes, e.g., improved pharmacological characteristics, while increasing or at least retaining substantially all of the biological activity of the unmodified peptide. For instance, the peptides can be modified by extending, decreasing the amino acid sequence of the peptide. Substitutions with different amino acids or amino acid mimetics can also be made.

The individual residues of the immunogenic peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the α-carbon, amide carbonyl, complete replacement of the amide bond, extensions, deletions or backbone cross-links. See, generally, Spatola, Chemistry and Biochemistry of Amino Acids, Peptides and Proteins, Vol. VI (Weinstein ed., 1983). Several peptide backbone modifications are known, these include, ψ [CH₂S], ψ [CH₂NH], ψ [CSNH₂], ψ [NHCO], ψ [COCH₂] and ψ [(E) or (Z) CH═CH]. The nomenclature used above, follows that suggested by Spatola, above. In this context, ψ indicates the absence of an amide bond. The structure that replaces the amide group is specified within the brackets.

Amino acid mimetics may also be incorporated in the peptides. An “amino acid mimetic” as used here is a moiety other than a naturally occurring amino acid that conformationally and functionally serves as a substitute for an amino acid in a peptide of the present invention. Such a moiety serves as a substitute for an amino acid residue if it does not interfere with the ability of the peptide to elicit an immune response against the appropriate G250-protein-derived epitope. Amino acid mimetics may include non-protein amino acids, such as β, γ-, δ-amino acids, β-, γ-, δ-imino acids (such as piperidine-4-carboxylic acid) as well as many derivatives of L-α-amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

As noted above, the peptides employed in the subject invention need not be identical, but may be substantially identical, to the amino acid sequences of SEQ ID NO.'s 12 or 15. Therefore, the peptides may be subject to various changes, such as insertions, deletions, and substitutions, either conservative or non-conservative, where such changes might provide for certain advantages in their use. The peptides of the invention can be modified in a number of ways so long as they comprise a sequence substantially identical (as defined below) to an amino acid sequence of SEQ ID NO.'s 12 or 15.

Alignment and comparison of relatively short amino acid sequences (less than about 30 residues) is typically straightforward. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of sequence similarity over the comparison window) generated by the various methods is selected.

The term “sequence identity” means that two polypeptide sequences are identical (i.e., on an amino acid-by-amino acid basis) over a window of comparison. The term “percentage of sequence identity” is calculated by comparing two optimally aligned sequences over the window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

As applied to the peptides of the invention, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and typtophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine.

Preferably, the peptides of the invention are such that—upon suitable administration to the body of a human or animal (mammal), e.g. as described hereinbelow—they are capable of generating or eliciting an immune response in the human or animal, against at least the peptide of the invention. Preferably, this immune response is a “significant” immune response, by which herein is generally meant a response that leads to a detectable change in the body of the human or animal to which the peptide of the invention is administered. Usually, this will be a detectable immune response against the peptide, such as the generation of antibodies against the peptide or more preferably a cellular immune response that occurs when a body—or a part, organ or tissue thereof—is exposed to an antigen.

Usually such an significant immune response will not only be directed only against the peptides of the invention, but also against proteins or peptides that contain such the relevant epitope of the invention as part of their amino acid sequence, as well as against structures, cells or tissues that contain, carry or express—e.g. on their surface—peptides or proteins containing such epitopes, such as the cells of the tumor to be treated.

The significant immune response elicited by the peptide of the invention may e.g. be determined using an immunological assay or an immunological detection technique known per se. Such an assay or detection technique may e.g. be an antigen-based assay or detection technique, in which for instance the peptide—or its relevant epitope—of the invention maybe used as the antigen. Examples of suitable immunological assays or detection techniques include, but are not limited to, blotting techniques such as Western blotting, ELISAs and RLAs, etc., for which reference is made to the standard handbooks (see e.g. Harlow and Lane, 1988, “Antibodies: A Laboratory Manual”, Cold Spring Harbor Laboratory, Cold Spring Harbor Laboratory Press, New York) as well as for instance WO 93/18152.

The assay or detection technique is usually applied to a suitable biological sample or fluid obtained from the patient, such as blood, lymph fluid and/or a tissue sample, including but not limited to a sample obtained from/through a biopsy. Such a sample may for instance be obtained after suitable administration of the peptide, e.g. as described below. The results obtained for this sample may then be compared to results obtained—i.e. using the same immunological assay—for a similar sample obtained prior to administration of the peptide, to determine whether a significant immune response has been generated or not. The assay or detection technique may also be a quantitative technique, providing comparative data on the immune response generated by different peptides of the invention.

More preferably, the peptides of the invention are such that they provide a therapeutically effective immune response, by which is meant an immune response that can attack or destroy a tumor present in the body of a patient, or at least prevent or limit the (further) growth and/or the spread of a tumor (i.e. within the same part or organ of the body and/or to other parts or organs of body), including but not limited to recidivism and/or metastasis.

Pharmaceutical and Other Compositions and Their Administration

In a further aspect, the invention relates to a pharmaceutical composition comprising a peptide of the invention as defined above. The pharmaceutical composition preferably at least comprises the peptide of the invention and a pharmaceutically acceptable carrier as described herein below. More preferably, the pharmaceutical composition is a vaccine which further preferably comprises an adjuvant as defined herein below.

In another aspect, the invention relates to a composition comprising an antigen-presenting cell as herein defined below, wherein the antigen-presenting cell is loaded with a peptide of the invention as defined above. The composition preferably is a pharmaceutical composition. Preferably the antigen-presenting cell is a dendritic cell, of which human antigen presenting cells are most preferred.

The peptides of the present invention and pharmaceutical compositions thereof are useful for administration to mammals, particularly humans, to treat and/or prevent a cancer expressing a G250 protein Suitable formulations are found in Remington's Pharmaceutical Sciences, Mack Publishing Company, Philadelphia, Pa., 17th ed. (1985), which is incorporated herein by reference.

The pharmaceutical compositions are intended for parenteral, oral or transdermal administration. Preferably, the pharmaceutical compositions are administered parenterally, e.g., subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, and triethanolamine oleate.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%75%. As noted above, the compositions are intended to induce an immune response to the peptides. Thus, compositions and methods of administration suitable for maximizing the immune response are preferred. For instance, peptides may be introduced into a host, including humans, linked to a carrier or as a homopolymer or heteropolymer of active peptide units. Alternatively, the a “cocktail” of peptides can be used. A mixture of more than one peptide has the advantage of increased immunological reaction and, where different peptides are used to make up the polymer, the additional ability to induce antibodies to a number of epitopes. For instance, peptides comprising sequences from hypervariable regions of α and β chains may be used in combination. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like.

The compositions preferably also include an adjuvant. A number of adjuvants are well known to one skilled in the art. Suitable adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic peptide. A particularly useful adjuvant and immunization schedule are described in Kwak et al New Eng. J. Med. 327-1209-1215 (1992), which is incorporated herein by reference. The immunological adjuvant described there comprises 5% (wt/vol) squalene, 2.5% Pluronic L121 polymer and 0.2% polysorbate in phosphate buffered saline.

The concentration of immunogenic peptides of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990). Both of these references are incorporated herein by reference in their entirety. E.g. transdermal delivery systems include patches, gels, tapes and creams, and can contain excipients such as solubilizers, permeation enhancers (e.g. fatty acids, fatty acid esters, fatty alcohols and amino acids), hydrophilic polymers (e.g. polycarbophil and polyvinyl pyrillidine and adhesives and tackifiers (e.g. polyisobutylenes, silicone-based adhesives, acrylates and polybutene). Transmucosal delivery systems include patches, tablets, suppositories, pessaries, gels, and creams, and can contain excipients such as solubilizers and enhancers (e.g. propylene glycol bile salts and amino acids), and other vehicles (e.g. polyethylene glycol, fatty acid esters and derivatives, and hydrophilic polymers such as hydroxypropylmethyl-cellulose and hyaluronic acid). Injectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone. Other delivery systems that can be used for administering the pharmaceutical composition of the invention include intranasal delivery systems such as sprays and powders, sublingual delivery systems and systems for delivery by inhalation. For administration by inhalation, the pharmaceutical compositions of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the peptides of the invention and a suitable powder base such as lactose or starch. The pharmaceutical compositions of the invention may be further formulated for administration by inhalation as e.g. described in U.S. Pat. No. 6,358,530.

The peptides of the invention can also be expressed by attenuated viral hosts, such as vaccinia or fowlpox. This approach involves the use of vaccinia virus as a vector to express nucleotide sequences that encode the peptides of the invention. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848, incorporated herein by reference. Another vector is BCG (Bacille Calmette Guerin). BCG vectors are described in Stover et al. (Nature 351:456-460 (1991)) which is incorporated herein by reference. A wide variety of other vectors useful for therapeutic administration or immunization with the peptides of the invention, e.g., Salmonella typhi vectors and the like, will be apparent to those skilled in the art. These include e.g. (mucosal) bacterial host containing nucleic acid that encoded the peptides of the invention and capable of expressing the peptides upon, usually oral administration, in the human or animal gastrointestinal tract. Such bacterial hosts include e.g. Salmonella typhi or Lactobacilli. Thus, included in the invention are compositions comprising DNA vaccines, gene therapy vectors, viruses or bacteria comprising nucleic acids encoding the peptides of the invention and that may be used in the methods of the invention to treat or prevent a cancer expressing a G250 protein.

Yet another method for administering of the peptide of the invention comprises administration in conjunction with an antigen presenting cell, by which is meant that the peptide is administered while carried by, attached to or otherwise associated with a suitable antigen presenting cell. For this purpose, a suitable antigen presenting cell may be “loaded/primed” in vitro with a peptide of the invention, preferably prior to administration, e.g. by adding the peptide of the invention to a composition comprising antigen presenting cells, such as e.g. an in vitro culture of antigen presenting cells. The antigen presenting cells thus loaded with the peptide of the invention may then be administered to the body of a patient, e.g. via intradermal injection, upon which they may (further) elicit an immune response against the peptide, e.g. by presenting the peptide to T-cells, Preferably, in this embodiment, autologous antigen presenting cells are used, meaning that the antigen presenting cells have been obtained from—or derived from cells obtained from—the patient to which they will be returned in loaded form. Preferably, the antigen presenting cells are dendritic cells. Preferably, the antigen presenting cells or dendritic cells are human cells. Methods for obtaining or deriving antigen presenting cells or dendritic cells are known in the art from e.g. handbooks like Coligan et al., 1994, In: Coico R, ed. Current protocols in immunology. Vol. 2: John Wiley & Sons, Inc., Chapter 7: Immunologic studies in humans and pages 7321-7326.

In another aspect the invention relates to a method for producing a pharmaceutical composition comprising the (poly)peptides of the invention. The method comprises at least the steps of mixing the (poly)peptides of the invention obtained in the methods described above with a pharmaceutically acceptable carrier and further constituents like adjuvant as described above.

Therapeutic Uses and Methods

In a further aspect, the invention relates to a use of a peptide of the invention as defined above, for the manufacture of a composition for the treatment or prevention of cancer. Preferably, the cancer is renal cell carcinoma, or a cancer of the kidney, the prostate, the head, the neck, the gastrointestinal tract or any part thereof, or the bladder. More preferably, the cancer is renal cell carcinoma, or a cancer of the colon, stomach or bladder. Most preferably, however, the invention relates to a use of a peptide of the invention as defined above, in the preparation of a composition for the treatment or prevention of a tumor that expresses a protein having the amino acid sequence with at least 95% identity SEQ ID NO. 16, or that expresses an immunogenic part of the protein.

In yet another aspect, the invention relates to a method for the treatment or prevention of a cancer in a subject the method comprising the administration to the subject of a composition, as defined above, in an amount effective to treat or prevent the cancer. Preferably, the composition is a pharmaceutical composition. Preferably, the cancer is renal cell carcinoma, or a cancer of the kidney, the prostate, the head, the neck, the gastrointestinal tract or any part thereof, or the bladder. More preferably, the cancer is renal cell carcinoma, or a cancer of the colon, stomach or bladder. Most preferably, however, the invention relates to a method for the treatment or prevention of a tumor in a subject, the method comprising the administration to the subject of a composition as defined above, in an amount effective to treat or prevent the tumor, and whereby the tumor is a tumor that expresses a protein having the amino acid sequence with at least 95% identity SEQ ID NO.16, or that expresses an immunogenic part of the protein.

The immunogenic peptides of the invention are administered prophylactically or to an individual already suffering from the disease. The compositions are administered to a patient in an amount sufficient to elicit a therapeutically effective (or significant) immune response as defined above. An amount adequate to accomplish this is defined as “therapeutically effective dose” or “immunogenically effective dose.” Amounts effective for this use will depend on, e.g., the peptide composition, the manner of administration, the stage and severity of the disease being treated, the weight and general state of health of the patient, and the judgement of the prescribing physician. Generally, treatment of a patient usually involves one or more administrations (also referred to as “vaccinations”) of a peptide (or composition) of the invention of the invention, in an amount sufficient for an immune response against the peptide to be generated. Preferably, such a treatment will involve one or more “priming” administrations of the peptide (or composition), followed by one or more “booster” administrations of the peptide (or composition), the latter usually after a suitable period of time after priming, and—if several booster administrations are used—separated by suitable time intervals.

Depending on the peptide (or composition) used, these will usually be in the range for the initial or priming immunization(s) (that is for therapeutic or prophylactic administration) from about 0.01 mg to about 1.0 mg per 70 kilogram patient, more commonly from about 0.1 mg to about 0.75 mg per 70 kg of body weight Boosting dosages are typically from about 0.05 mg to about 0.75 mg of peptide per 70 kilogram patient, more commonly from about 0.1 mg to about 0.5 mg per 70 kg of body weight, using a boosting regimen over weeks to months depending upon the patient's response and condition. A suitable protocol would include injection at time 0, 2, 6, 10 and 14 weeks, followed by booster injections at 24 and 28 weeks. Alternatively, a suitable administration regimen may involve a first priming administration on day one, optionally followed by one or more further priming administrations in the next 8 weeks, in which said administrations may for instance be separated by 14 to 28 days or more. These priming administrations may then be followed by one or more booster administrations, e.g. in weeks 8 to 52, which booster administrations will usually be separated by 14 to 28 days or more. Usually, the one or more priming and the one or more booster administrations are administered via the same route of administration (e.g. subcutaneously and/or intradermally) and using the same (type of) pharmaceutical formulation for the peptide. The invention encompasses both the use of only a single species of the peptide of the invention, as well as the use of any suitable combination of the different peptides of the invention, in any suitable manner.

In a preferred embodiment of the invention, the method for the treatment or prevention of a cancer in a subject is applied in a MHC class I and/or II haplotype specific manner. Thus, the MHC class I and/or II haplotypes of the subject are determined and the method is preferably applied to a subject whose MHC class I and/or II haplotypes recognize the peptide of the invention. Such preferred MHC class I and/or II haplotypes are selected from HLA-A2.1 and/or HLA-DR, of which preferably, HLA-DR3, HLA-DR4 or HLA-DR11 or combinations thereof. Similarly, the peptides of the invention may preferably be used for the manufacture of a composition for the treatment or prevention of a cancer in a MHC class I and/or II haplotype specific manner.

When a peptide of the invention is administered in conjunction with an antigen presenting cell, i.e. as described above, the cells carrying the peptide may be administered in a manner known per se for the administration of cells to the body of a patient. Again, this administration is preferably such that it results in an immune response, in particular a significant immune response, and preferably a therapeutically effective immune response. The cells carrying the peptide may e.g. be administered via intradermal, intravenous or intra-arterial injection.

For this purpose, the cells are usually provided in the form of a cell suspension in a suitable liquid medium, which is most preferably pharmaceutically effective and which may be the same as the medium used for maintaining, cultivating and/or loading/priming the cells in vitro. Examples are physiological solutions, such as phosphate-buffer-salt-solutions. In such cell preparations for administration, the peptide-loaded cells will usually be present in amounts/concentrations of 10²-10⁸ cells/ml, preferably 10⁴-10⁵ cells/ml. Alternatively, the cells may for instance be administered in the form of apoptotic bodies.

The cells may be administered once or several times, for instance according to a regimen involving priming and boosting, e.g. essentially as described above, although the invention is not limited thereto. In doing so, the cells are preferably administered in suitable amounts, i.e. such that an immune response, in particular a significant immune response, and preferably a therapeutically effective immune response, is obtained. The specific amount(s) to be administered will usually be determined by the clinician on the basis of the description given herein, taking into account such factors as the tumor to be treated, the general condition of the patient, the cells and/or peptides used, the type of vaccination (e.g. priming or boosting) and the desired administration regimen.

Furthermore, although not preferred, the invention also encompasses the administration of fractions, lysates and/or fragments that are obtained from cells that have been the loaded/primed in vitro with the peptide, and in particular the cells described above. In particular those fractions, lysates or fragments may be used which when administered in a suitable manner, in suitable amounts and/or according to a suitable regimen, e.g. as described above—can provide a significant immune response, and preferably a therapeutically effective immune response. Such cell fractions, lysates or fragments may be in the form of, or incorporated into, a pharmaceutical preparation, which essentially may be as described above for the peptides of the invention.

Finally, precursors of the peptides of the invention may be used. A precursor of a peptide of the invention is herein understood to mean any molecule which upon processing is capable of producing a peptide of the invention. Precursors of the peptides of the invention will preferably be such that—when administered in a suitable manner, in suitable amounts and/or according to a suitable regimen, e.g. as described above—they can provide a significant immune response, and preferably a therapeutically effective immune response. These precursors may again be formulated and administered essentially as described above for the peptides of the invention.

Gene Therapy

Yet another aspect of the invention relates to a gene therapy agent comprising a nucleotide sequence that encodes a peptide of the invention as defined above, and optionally one or more further elements of gene therapy agents known per se. The invention thus discloses nucleotide sequences that encode peptides of the invention as defined above, and that may be used in the manufacture of a composition for the treatment of a cancer by gene therapy. In a further aspect, the invention relates to a method for the treatment or prevention of a cancer in a subject, the method comprising the administration to the subject of a gene therapy agent—comprising a nucleotide sequence that encodes a peptide of the invention as defined above, and optionally one or more further elements of gene therapy agents known per se—, in an amount effective to treat or prevent the cancer.

The gene therapy agent preferably is used to generate—by suitable administration to a human or animal (mammal)—a “significant” immune response as described above, and preferably a “protective” immune response as described above; which response is essentially similar to the significant/protective immune response that can be induced by administration of a peptide of the invention as described above. Such a gene therapy agent will usually be such that, upon administration, it will allow or provide for expression of a peptide of SEQ ID NO. 12—or a variant or analogue thereof as defined above—in the body of the human or animal, or in any part, organ, tissue or cell of such a human or animal, including (the cells of) the tumor to be treated. The expression of the peptide provided by the gene therapy agent should further be such that the expressed peptide can come into contact with an antigen-presenting cell as defined above, or otherwise can come into contact with cells involved in the immune system, so as to generate a significant, and preferably a protective, immune response against (at least) the peptide. Usually, such a gene therapy agent of the invention will comprise a single or double stranded nucleotide sequence (e.g. a DNA or RNA sequence), which at least comprises a nucleotide sequence that codes for a peptide of the invention. Usually, such a gene therapy agent will be in the form of a suitable vector—e.g. a viral vector such as an adenoviral vector—which at least encodes a peptide of the invention Such a vector may contain all other elements for gene therapy vectors known per se, including but not limited to genetic elements such as a suitable promoter, a suitable terminator or other regulatory elements operably linked to the peptide-encoding sequence; as well as integration factors or other elements that allow for the gene therapy agent to enter into and be expressed in the cell, e.g. by integration into the (genomic) DNA present in the cell. Such a vector may also be suitably “packaged”, e.g. with one or more suitable capsid proteins, to provide a viral particle. Also, such a gene therapy agent may express the peptide of the invention as part of a larger amino acid sequence (e.g. a protein or polypeptide), or as a fusion with one or more further peptide sequences.

The gene therapy agent may be administered in a manner known per se, e.g. by exposing the body of the human or animal—or any part, organ, tissue or cell of the human or animal, including (the cells of) the tumor to be treated—to the gene therapy agent. Also, such a gene therapy agent may be used in vitro to infect suitable cells—such as cells derived from the patient and/or of the tumor to be treated—upon which these cells may then be introduced to the body of the human or animal, to provide the desired significant immune response, and preferably a protective immune response, as defined hereinabove.

The gene therapy agent may also be formulated in a manner known per se, e.g. to provide a pharmaceutical preparation (gene therapy preparation), e.g. using one or more pharmaceutically acceptable carriers, adjuvants, and/or excipients. Such preparations form a further aspect of the invention.

Other Applications of the Peptides of the Invention

Besides the above therapeutic applications, the peptides of the invention may also be used as antigens representative of the protein of SEQ ID NO. 16 or variants thereof, e.g. in immunological, diagnostic and/or analytical applications, such as those described e.g. in WO 93/18152. For this purpose, the peptides of the invention may also form part of a kit, e.g. in combination with other components for immunological, diagnostic and/or analytical kits.

### DESCRIPTION OF THE FIGURES

FIG. 1. T-helper cell lines I and III specifically proliferate in response to G250-derived peptides. Autologous EBV-B cells loaded with an irrelevant pool (open columns) and the relevant pool (closed columns) of G250-derived peptides were used as stimulator cells in a proliferation assay. From 1 donor, 3 CD4⁺ T-cell cultures, induced against autologous DC's loaded with 1 of the 3 groups of G250-derived peptides, were used as responder cells.

FIG. 2. T-helper cell line III recognizes peptide G250:249-268 in the context of HLA-DR. (a) The G250-derived peptide recognized by T-helper cell line III was examined using a proliferation assay in which EBV-B cells were loaded with each peptide of pool III separately. (b) To identify the MHC class II molecule by which peptide G250:249-268 is presented to T-helper cell line III, peptide-specific IFN-Y production was measured in the presence or absence of blocking antibodies against HLA-DR (L243), HLA-DP (B7/21) and HLA-DQ (TY22).

FIG. 3. Naturally processed G250 is recognized by T-helper cell line III. Peptide G250:249-268-specific T-helper cells were tested for their ability to proliferate (a) and to secrete IFN-Y (b) in response to autologous DC's loaded with rhodopsin protein (containing 25% baculovirus proteins) or autologous DC's loaded with G250 protein (>95% pure). One of 3 representative experiments is shown. MHC class II restriction was examined by blocking G250-specific proliferation with anti-HLA-DR antibody (L243) and both anti-HLA-DP (B7/21) and anti-HLA-DQ (TY22) (a). One of 2 representative experiments is shown.

### EXAMPLES

1. Materials and Methods

1.1. Proteins, Lysates and Peptides

G250 protein (>95% pure) and rhodopsin protein (75% pure) were purified from Spodoptera fugiperda (Sf9) cells (ATCC, Rockville, Md.) infected with G250 baculovirus and rhodopsin baculovirus, respectively, as described (Grabmaier et al., 2000, supra; Janssen et al., 1995, J. Biol Chem. 270; 11222-11229). Peptides were synthesized by Fmoc chemistry using a multiple-peptide synthesizer (J. W. Dryfhout, Leiden University Medical Center, Leiden, The Netherlands). As determined by reversed-phase HPLC, peptides were >90% pure.

1.2, Induction of CD4⁺ T Cells Using Peptide-Loaded DCs

At day −8, PBMCs of healthy individuals were separated using Percoll-density centrifugation and allowed to adhere for 1 hr at 37° C. in RPMI-1640 (Life Technologies, Grand Island, N.Y.) enriched with 2% human serum in 75 cm² tissue culture flasks (Costar, Badhoevedorp, the Netherlands). Adherent monocytes were cultured in X-VIVO 15 medium (BioWhittaker, Walkersville, Md.) enriched with 1% autologous serum in the presence of IL-4 (500 U/ml) and GM-CSF (800 U/ml; both from Schering-Plough, Amstelveen, the Netherlands) for 6 days. Fresh cytoline-containing culture medium was added at day −5. At day −2, immature DCs were stimulated with 10 ng/ml TNF-α (Bender, Vienna, Austria) and 10 μg/ml prostaglandin E₂ (Sigma, St. Louis, Mo.). Based on SYFPEITHI and TEPITOPE, 2 MHC class II-restricted epitope prediction software programs (kindly performed by Dr. S. Stevanovic; for references see: Rammensee et al., 1999, Immunogenetics 50: 213-219; de Lalla et al., 1999, J. Immunol. 163: 1725-1729; Sturniolo et al., 1999, Nat. Biotechnol. 17: 555-561) 14 G250-derived peptides were selected (Table 2). At day −1, 10 μg/ml of each G250-derived peptide were added to the DCs (4 or 5 peptides/DC pool). At day 0, peptide-loaded mature DCs were loaded again with 10 μg/ml of each G250-derived peptide at 37° C. for 4 hr. Peptide-loaded DCs (5×10⁴/well) were co-cultured with 5×10⁵ enriched autologous CD4⁺ T cells [depleted for CD8⁺ and CD56⁺ cells by magnetic sorting (Dynal, Oslo, Norway)] in X-VIVO 15 medium supplemented with 1% autologous serum in the presence of 1,000 U/ml IL-6 (Novartis, Basel, Switzerland) and 10 ng/ml IL-12 (R&D Systems, Abingdon, UK). At days 7 and 14, responder T cells were restimulated with peptide-loaded immature DCs (37° C., 4 hr) and 20 IU/ml IL-2 (Chiron, Berkeley, Calif.). At days 21 and 35, bulk T cells were tested for peptide specificity in both a proliferation assay and IFN-γ secretion assay with peptide-loaded autologous EBV-B cells as stimulator cells. Additionally, the percentage of CD4⁺ T cells in these T-cell cultures was established by indirect immunofluorescence using mouse antihuman CD4 MAb RIV-7 (Leerling et al., 1990, Dev Biol. Stand. 71: 191-200) and FITC-labeled goat antimouse secondary antibodies (Zymed, San Francisco, Calif.) followed by flow cytometry (FACScan; Becton Dickinson, Mountain View, Calif.). Every 7 days, T cells were alternately given 20 IU/ml IL=2 or restimulated with peptide-loaded EBV-B cells (37° C., 4 hr) and 20 IU/ml IL-2.

1.3. IFN-γ Release Assay and Proliferation Assay

Autologous APCs were loaded with G250-derived peptides at 37° C. for 4 hr Antigen-loaded APCs were irradiated (5,500 rad) and plated in 96-well round-bottomed plates (Costar) at 3.5×10⁵ cells/well in X-VIVO 15 medium enriched with 1% autologous serum. Bulk T cells were added at 5×10⁵ cells/well. For HLA-blocking experiments, antibodies B7/21 (anti-HLA-DP), TY22 (anti-LA-DQ) and L243 (anti-HLA-DR) (antibodies kindly provided by Dr. G. Pawelec) were added to each well with an end concentration of 25% (v/v); at this concentration, proliferation could completely be blocked. To test whether T-cell cultures were able to release IFN-γ upon antigen-specific stimulation, the supernatants of these cultures were harvested after 16 hr. Subsequently, the amount of IFN-γ in the supernatants was determined using an IFN-γ-specific sandwich ELISA. Proliferation of responder T cells was determined after 72 hr of culture by pulsing the cells for another 16 hr with 1 μCi/well [³H]TdR (Amersham, Aylesbury, UK). Thymidine incorporation was measured using a liquid scintillation counter (LKB Wallac, UK).

TABLE 2
G250 derived peptides (20-mer) covering predicted
HLA class II-binding peptides (*overlapping amino
acids are depicted in bold).

| Position in G250 | | | SEQ |
|---|---|---|---|
| protein sequence | Amino acid sequence* | Group | ID NO. |
| 146-165 | GDPPWPRVSPACAGRFQSPV | I | 1 |
| 154-173 | SPACAGRFQSPVDIRPQLAA | I | 2 |
| 162-181 | QSPVDIRPQLAAFCPALRPL | I | 3 |
| 170-189 | QLAAFCPALRPLELLGFQLP | I | 4 |
| 178-197 | LRPLELLGFQLPPLPELRLR | I | 5 |
| 399-418 | AAEPVQLNSCLAAGDILALV | II | 6 |
| 407-426 | SCLAAGDILALVFGLLFAVT | II | 7 |
| 415-434 | LALVFGLLFAVTSVAFLVQM | II | 8 |
| 423-442 | FAVTSVAFLVQMRRQHRRGT | II | 9 |
| 105-124 | EGSLKLEDLPTVEAPGDPQE | III | 10 |
| 241-260 | VEGHRFPAEIHVVHLSTAFA | III | 11 |
| 249-268 | EIHVVHLSTAFARVDEALGR | III | 12 |
| 336-355 | AQGVTWTVFNQTVMLSAKQL | III | 13 |
| 344-363 | FNQTVMLSAKQLHTLSDTLW | III | 14 |

2. Results

2.1 Induction of a G250-Derived, Peptide-Specific T-Helper Response

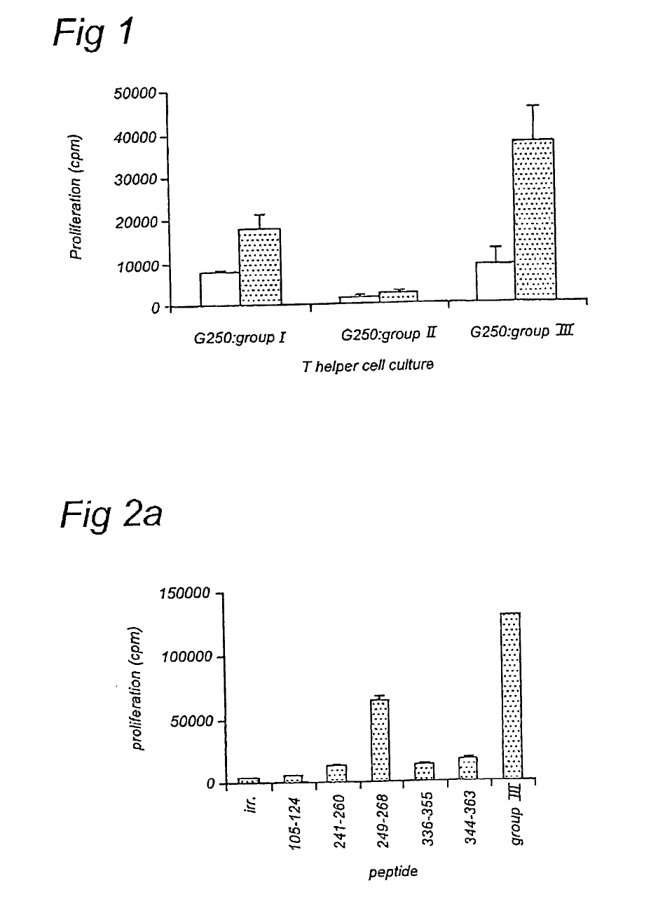

Antitumor reactivity of CTLs is enhanced by antigen-specific T-helper responses. To investigate the potential of the RCC-associated antigen G250 to induce a CD4⁺ T-cell response, we selected 14 G250-derived peptides based on 2 prediction software programs, SYFPEITHI and TEPITOPE. The selected 20 mer peptides were located in regions of the G250 protein that contained a high density of the predicted binding motifs for HLA-DR1, HLA-DR3, HLA-DR4 and HLA-DR11. For the induction of anti-G250 T-helper cells, we used professional antigen-presenting DCs from healthy individuals. Per donor, 3 pools of DCs were loaded separately with 1 of the 3 groups, each containing 4 or 5 G250-derived peptides (Table 2). Peptide-loaded DCs were cocultured with autologous CD4⁺ T cells. As shown in FIG. 1, we obtained T-cell lines that specifically proliferated upon stimulation with autologous EBV-B cells loaded with G250-derived peptides from groups I and III. In contrast, out of 8 healthy donors, no peptide-specific T-cell proliferation was obtained against peptides of group II. Since the T-cell line raised against G250-derived peptides of group III exhibited the highest proliferative response and could be expanded most efficiently, these T cells were subjected to further analysis.

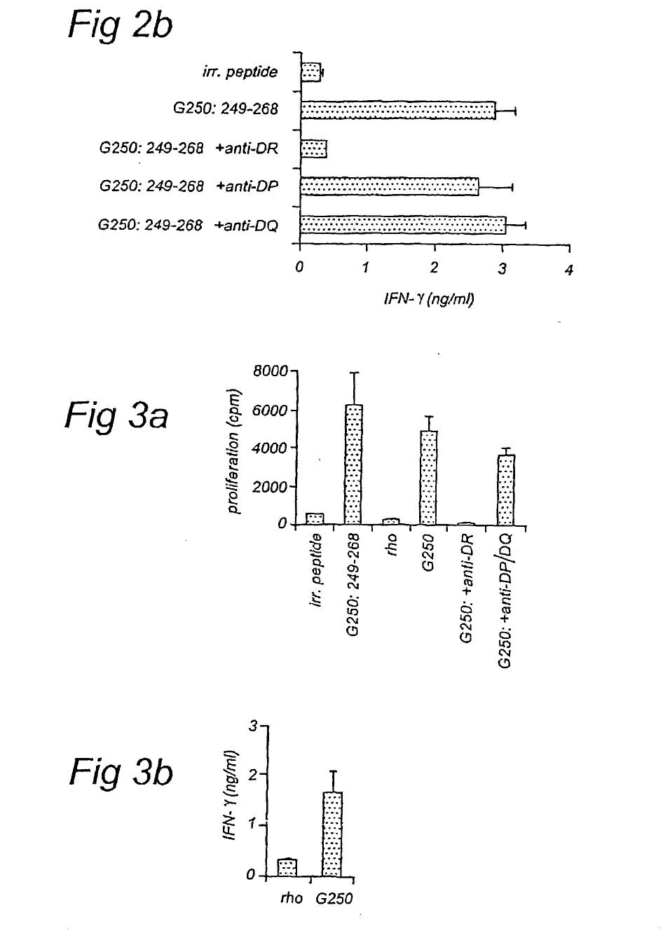

To test which G250-derived peptides of group III were recognized by the T-helper cell line III, autologous EBV-B cells loaded with each G250-derived peptide of group III separately were used as stimulator cells in a proliferation assay. FIG. 2a shows that this T-cell culture (>98% CD4⁺) specifically recognized peptide G250:249-268 and none of the other G250-derived peptides. Subsequently, the MHC class II molecule by which peptide G250:249-268 is presented to T cells was examined. FIG. 2b shows that peptide G250:249-268 specifically induced secretion of IFN-γ-by T-helper cell line III and that IFN-γ production was blocked with an antibody against HLA-DR (L243) but not by antibodies against HLA-DP (B7/21) or HLA-DQ (TY22). These results show that recognition of peptide G250:249-268 by T-helper cell line III is HLA-DR-restricted.

2.2. G250:249-268-Specific T-Helper Cell Line Recognizes Naturally Processed G250

To determine whether G250-derived peptide 249-268 is naturally processed and presented, autologous DCs were loaded with purified G250 protein (5 μg/ml) and used to stimulate CD4⁺ T-helper cell line III For this purpose, baculovirus-produced G250 protein was used and recombinant rhodopsin, produced in the same baculovirus system, included as a negative control protein. T cells specifically proliferated (FIG. 3a) and specifically secreted IFN-γ (FIG. 3b) upon interaction with autologous DCs loaded with G250 protein but not upon interaction with autologous DCs loaded with rhodopsin protein. Since the fraction of rhodopsin protein contains 25% baculovirus proteins, the observed proliferation and IFN-γ secretion are G250-specific. As shown in FIG. 3a, recognition of naturally processed G250 could be blocked by anti-HLA-DR antibodies but not by anti-HLA-DP and anti-HLA-DQ antibodies. These data demonstrate that peptide 249-268 is naturally processed from the G250 protein and presented by HLA-DR.

1. A peptide other than the human G250 protein, whereby the peptide comprises the amino acid sequence of SEQ ID NO. 15 or an amino acid sequence with at most 3 amino acid replacements with respect to the amino acid sequence of SEQ ID NO. 15.

2. A peptide according to claim 1, whereby the peptide consists of the amino acid sequence of SEQ ID NO. 15 or an amino acid sequence with at most 3 amino acid replacements with respect to the amino acid sequence of SEQ ID NO. 15.

3. A peptide according to claim 1, whereby the peptide comprises the amino acid sequence of SEQ ID NO. 12 or an amino acid sequence with at most 4 amino acid replacements with respect to the amino acid sequence of SEQ ID NO. 12.

4. A peptide according to claim 3, whereby the peptide consists of the amino acid sequence of SEQ ID NO. 12 or an amino acid sequence with at most 3 amino acid replacements with respect to the amino acid sequence of SEQ ID NO. 12.

5. A peptide according to any one of claims 1-4, whereby the amino acid replacements are conservative replacements, preferably selected from the group consisting of the amino acid replacements in the amino acid sequence of SEQ ID NO.12: L, P, A, F, W or M in position 4; M in position 7; and L, P, A, F, W or M in position 14.

6. A pharmaceutical composition comprising a peptide as defined in any one of claims 1-5 and a pharmaceutically acceptable carrier.

7. A pharmaceutical composition according to claim 6, whereby composition is a vaccine and further comprises an adjuvant.

8. A composition comprising an antigen presenting cell, wherein the antigen-presenting cell is loaded with a peptide as defined in any one of claims 1-5.

9. A composition according to claim 8, wherein the antigen presenting cell is a dendritic cell.

10. A composition according to claims 8 or 9, wherein the antigen presenting cell is a human cell.

11. Use of a peptide as defined in any one of claims 1-5 for the manufacture of a composition for the treatment or prevention of cancer.

12. A use according to claim 11, wherein the cancer is renal cell carcinoma, or a cancer of the kidney, the prostate, the head, the neck, the gastrointestinal tract or any part thereof, or the bladder.

13. A use according to claim 12, wherein the cancer is renal cell carcinoma, or a cancer of the colon, stomach or bladder.

14. A use of a peptide as defined in any one of claims 1-5, in the preparation of a composition for the treatment or prevention of a tumor that expresses a protein having the amino acid sequence with at least 95% identity SEQ ID NO.16, or that expresses an immunogenic part of the protein.

15. A method for the treatment or prevention of a cancer in a subject, the method comprising the administration to the subject of a composition as defined in any one of claims 6-10, in an amount effective to treat or prevent the cancer.

16. A method according to claim 15, wherein the cancer is renal cell carcinoma, or a cancer of the kidney, the prostate, the head, the neck, the gastrointestinal tract or any part thereof, or the bladder.

17. A method according to claim 16, wherein the cancer is renal cell carcinoma, or a cancer of the colon, stomach or bladder.

18. A method for the treatment or prevention of a tumor in a subject, the method comprising the administration to the subject of a composition as defined in any one of claims 6-10, in an amount effective to treat or prevent the tumor, and whereby the tumor is a tumor that expresses a protein having the amino acid sequence with at least 95% identity SEQ ID NO. 16, or that expresses an immunogenic part of the protein.

19. A gene therapy agent, comprising a nucleotide sequence that encodes a peptide as defined in any one of claims 1-5, and optionally one or more further elements of gene therapy agents known per se.

20. Use of a nucleotide sequence that encodes a peptide as defined in any one of claims 1-5 in the manufacture of a composition for the treatment of a cancer by gene therapy.

21. A method for the treatment or prevention of a cancer in a subject, the method comprising the administration to the subject of a gene therapy agent as defined in claim 19, in an amount effective to treat or prevent the cancer.


SEQUENCE LISTING (16 sequences)

SEQ ID NO: 1 - protein, 20 aa, Homo sapiens
GDPPWPRVSPACAGRFQSPV

SEQ ID NO: 2 - protein, 20 aa, Homo sapiens
SPACAGRFQSPVDIRPQLAA

SEQ ID NO: 3 - protein, 20 aa, Homo sapiens
QSPVDIRPQLAAFCPALRPL

SEQ ID NO: 4 - protein, 20 aa, Homo sapiens
QLAAFCPALRPLELLGFQLP

SEQ ID NO: 5 - protein, 20 aa, Homo sapiens
LRPLELLGFQLPPLPELRLR

SEQ ID NO: 6 - protein, 20 aa, Homo sapiens
AAEPVQLNSCLAAGDILALV

SEQ ID NO: 7 - protein, 20 aa, Homo sapiens
SCLAAGDILALVFGLLFAVT

SEQ ID NO: 8 - protein, 20 aa, Homo sapiens
LALVFGLLFAVTSVAFLVQM

SEQ ID NO: 9 - protein, 20 aa, Homo sapiens
FAVTSVAFLVQMRRQHRRGT

SEQ ID NO: 10 - protein, 20 aa, Homo sapiens
EGSLKLEDLPTVEAPGDPQE

SEQ ID NO: 11 - protein, 20 aa, Homo sapiens
VEGHRFPAEIHVVHLSTAFA

SEQ ID NO: 12 - protein, 20 aa, Homo sapiens
EIHVVHLSTAFARVDEALGR

SEQ ID NO: 13 - protein, 20 aa, Homo sapiens
AQGVIWTVFNQTVMLSAKQL

SEQ ID NO: 14 - protein, 20 aa, Homo sapiens
FNQTVMLSAKQLHTLSDTLW

SEQ ID NO: 15 - protein, 15 aa, Homo sapiens
EIHVVHLSTAFARVD

SEQ ID NO: 16 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA